(19) United States
(12) Patent Application Publication
(10) Pub. No.: US 2016/0338567 A1
(43) Pub. Date: Nov. 24, 2016

(54) Industrial Dishwasher

(71) Applicant: K-ONE INDUSTRIES PTE. LTD., Singapore (SG)

(72) Inventors: Mun Chew KONG, Singapore (SG); Chze Seong CHEONG, Singapore (SG)

(21) Appl. No.: 15/105,800

(22) PCT Filed: Dec. 18, 2014
(86) PCT No.: PCT/SG2014/000605

§ 371 Date: Jun. 17, 2016

(30) Foreign Application Priority Data
Dec. 20, 2013 (SG) 2013094628

(51) Int. Cl. A47L 15/10; A47L 15/24; A47L 15/13; A61L 2/18; A47L 15/00; A47L 15/48; C02F 1/461; A47L 15/42; A47L 15/06

(52) U.S. Cl. CPC A47L 15/10; A47L 15/4285; A47L 15/245; A47L 15/4234; A47L 15/13; A47L 15/06; A47L 15/0076; A47L 15/4278; A47L 15/4287; A47L 15/4295; A47L 15/486; A47L 15/0002; C02F 1/4618; A61L 2/18; A47L 2601/06; C02F 2001/46185; C02F 2001/4619; C02F 2103/02

(57) ABSTRACT

An industrial dishwasher includes a detergent station for washing serviceware with cleaning agent; a sanitising station for disinfecting the serviceware with sterilising agent; a neutralisation station connected to the detergent station, the sanitising station or both for deactivating the cleaning agent, the sterilising agent or both; and one or more conveyor chains connected to the detergent station, the sanitising station or both stations for carrying the serviceware to the detergent station, the sanitising station or both stations.

The present application relates to an Industrial dishwasher. The application also relates methods of making, assembling, disassembling, installing, modifying, configuring, upgrading, downgrading, simplifying, maintaining and using the industrial dishwasher.

Industrial dishwashers are widely used in inflight catering centres, hospitals and hotels where soiled food utensils, trays and tableware items (e.g. dishes or dishware, cutlery) are washed with large volumes at high speed. Cleaned food utensils, trays, cutlery and tableware items are often repeatedly used for food catering to flight passengers, patients and guests. Users or buyers of the industrial dishwashers typically desire to deploy the industrial dishwashers with high cleaning capacity and efficiency, but less resource consumption, such as on water, electricity, manpower and production floor area. However, traditional industrial dishwashers typically employ large amount of hot water (e.g. at about 72° C. or higher) and chemical detergent (i.e. mixture of surfactants with “cleaning properties” in dilute solutions) for cleaning soiled item effectively, thus incurring high cost and polluting environment.

The present invention(s) alms to provide one or more new and useful industrial dishwashers. The present invention also aims to provide one or more new and useful methods of making, assembling, disassembling, installing, modifying, configuring, upgrading, downgrading, simplifying, maintaining and using the industrial dishwasher. Essential features of the invention(s) are provided by one or more independent claims, whilst advantageous features of the invention(s) are given by their dependent claims. The present application claims priority of Singapore patent application 2013094628 with the title of Industrial Dishwasher, which was filed on 20 Dec. 2014. All content of this earlier application is hereby incorporated by reference.

According to a first aspect of the invention, the present application provides an industrial dishwasher that comprises a detergent station for washing serviceware with cleaning agent; a sanitising station for disinfecting the serviceware with a sterilising agent; a neutralisation station connected to the detergent station, the sanitising station or both for deactivating, deactivating, diluting, degrading, decomposing or reducing potency of the cleaning agent, the sterilising agent or both the cleaning agent and the sterilising agent by or at the neutralisation station. The Industrial dishwasher further comprises one or more conveyor chains that are connected to the detergent station, the sanitising station or both the detergent station and the sanitising station for carrying the serviceware to the detergent station, the sanitising station or both. The serviceware include items or tools used for food or beverage handling, consumption, packaging or transportation, such as trays, cutlery items, bowls, chop sticks and cups.

Here, the cleaning agent is also known as detergent which is a surfactant or a mixture of surfactants with “cleaning properties” in dilute solutions. The cleaning agent includes any of a group of synthetic, organic, liquid or water-soluble cleaning agents that are not prepared from fats and oils, are not inactivated by hard water, and have wetting-agent as well as emulsifying-agent properties. The cleaning agent (i.e. cleansing agent) or detergent is oil-soluble and capable of holding insoluble foreign matter in suspension, sometimes used in lubricating oils, dry-cleaning preparations, etc., including soap. The sterilising agent comprises antiseptics (e.g. bacterlocidal and bacteriostatic), antibacterial material(s) (e.g. microbicides) and disinfectants that can destroy or inhibit growth of bacteria, germs or fungus. Alternatively, the sterilising agent may merely comprises clean water alone or in combination with other fluid(s) for rinsing off detergent residue.

Although the cleaning agent and the sterilising agent are effective for cleaning serviceware (e.g. cutlery and dishes), used or depleted cleaning agent and the sterilising agent are often harmful to environment if discharged directly to drains. For example, the discharge of soluble phosphates into natural waters has led to problems with eutrophication, or the growth of living things in lakes and streams, often where it is not desirable. Australia began phasing out the use of phosphates in its detergents in year 2011, with an all-out ban expected to take effect in year 2014. In the present industrial dishwasher, the neutralisation station collects the cleansing/cleaning agent, the sterilising agent or both for their decomposition, degradation or dilution such that discharged washing fluid from the neutralisation station becomes more environmentally friendly. For example, aquatic fungi, aquatic bacterial flora, bacillus, bacillus cereus or other species of bacteria can be deposited at the neutralisation station regularly, intermittently, periodically or continuously for degrading or decomposing household detergents (surfactants) effectively. Therefore, the present industrial dishwasher is accordingly known as Green Dishwasher or Environmental Friendly Dishwasher. Alternatively, washing liquids from the detergent station or the sanitizing station are added with additional liquid(s) (e.g. water) or used liquid(s) (e.g. dirty water) such that waste water treated by or discharged from the neutralisation station possesses less potency for damaging the environment.

The one or more conveyor chains are configured to carry the serviceware (e.g. trays, mugs, glasses) through one or more of these washing stations (e.g. detergent station, sanitising station and neutralisation station) sequentially or in parallel such that the serviceware (i.e. serviceware items) can be washed more effectively or efficiently. Manual operation or ambient contamination is avoided because the industrial dishwasher can wash the serviceware automatically in a controlled or closed room.

Particularly, the one or more conveyor chains (also known as conveyor or conveyor belt) can be further extended or connect to the neutralisation station for rinsing the serviceware at the neutralisation station. Although neutralised fluid(s) at the neutralisation station may not be clean or pure, food particles or grease can still be purged off by strong currents of the neutralised fluid flowing inside a container (e.g. scraping container or prewashing container) of the neutralisation station. In other words, the soiled serviceware is better prepared for washing by the detergent station and/or sanitising station, after the initial cleaning at the neutralisation station.

The one or more conveyor chains may comprise an endless belt for moving the serviceware from the detergent station to the sanitising station. The conveyor chain is alternatively known as conveyor system, conveyor chain conveyor. The serviceware are secured to the conveyor chain and further carried through one or more of these washing stations. The conveyor chain or the endless belt may be partially or fully submerged in one or more of these stations for maximising contact between the serviceware and washing liquids. Accordingly, the one or more conveyor chains can be propelled and continuously used washing the serviceware.

The industrial dishwasher can further comprise an electrolyzer for supplying the cleaning agent to the detergent station, the sterilising agent to the sanitising station or both. The cleaning agent comprises alkali electrolysis water, whilst the sterilising agent comprises acid electrolysis water. Both the alkali electrolysis water and the acid electrolysis water can be produced by passing electric current through water dissolved with salt. The electrolyzer can be installed next to or near the detergent station or/and the sanitising station for immediate usage after their production for achieving high effectiveness. Hence, in other words, the electrolyzer can be configured to produce alkali electrolysis water at its positive electrode(s) as the cleaning agent, and acid electrolysis water at its negative electrode(s) as the sterilising agent. The alkali electrolysis water preferably has pH value from 11.0 to 12.0 for cleansing, whilst the sterilising agent advantageously possess pH value from 2.7 to 5.0 for bacteria elimination, deodorization and bleaching.

The Industrial dishwasher may further comprise a cavitation generator that is connected to the detergent station, the sanitising station, the neutralisation station or a combination of any of these stations for producing voids in one or more washing liquids in these stations. Tiny cavities or micro voids in the washing liquids can enhance cleaning abilities of the detergent (cleaning agent) and the sterilising agent such that less water or washing time is required for cleaning soiled dishes. Particularly, since the alkali electrolysis water and the acid electrolysis water typically deteriorate over time, the tiny cavities or micro voids in water can greatly prolong and stabilise potency and enhance effectiveness of the alkali electrolysis water and the acid electrolysis water, thereby reducing electricity (e.g. for heating) and water consumption (e.g. for rinsing) of the industrial dishwasher.

The cavitation generator can comprise a mixer for injecting one or more types of gases into the washing liquid in order to form micro or ultra-fine cavities. Although there are numerous ways of generating the tiny cavities or voids in liquid(s) (e.g. water), injecting air into the liquid(s) via small orifices under pressure is generally effective and requires low cost for production. The mixer effectively causes gases (e.g. air) and liquid (e.g. water) to clash onto each other in turbulence such that s transparent liquid (e.g. water) can become milky/cloudy in appearance, when saturated with the tiny cavities and voids. For industrial dishwashers, water impregnated with air cavities is highly oxidative, effective for cleaning.

In one embodiment, the mixer comprises a porous cylinder for guiding the gas and the liquid flowing along opposite sides of the porous cylinder in a whirling motion. Rotation of the gas and liquid can be achieved and confined in a compact container, which does not occupies large room on the industrial dishwasher. The mixer or the industrial dishwasher thus require less production floor area.

In another embodiment, the cavitation generator comprises an ultrasonic generator. The ultrasonic generator causes rapid formation and collapse of minute cavities in its surround liquid (e.g. water), known as cavitation. These minute cavities quickly increase in size till their implosion against surface of serviceware in the washing stations. Energy release of the implosion lifts contamination off inner most recesses of intricately shaped parts, such as from scratches of cutlery items.

The ultrasonic generator may comprise one or more ultrasonic transducers on a wall of the washing stations. These ultrasonic transducers create the tiny cavities in the washing stations effectively. In fact, the ultrasonic generator can comprise multiple ultrasonic transducers that are evenly distributed on a bottom side of the washing stations. The tiny cavities can thus rise slowly within the washing stations for reaching immersed serviceware with prolonged duration.

The detergent station can comprise a detergent container and a prewashing container. The detergent container and the prewashing container are closely or contiguously connected together for cascading used washing liquid from container to the other. Hence, each of the washing stations does not require fresh washing liquid supply (e.g. clean water, new cleaning agent, freshly prepared sterilising agent) because washing liquid(s) from a prior or subsequent washing station may be reused by a next/another washing station. Transferring of the used washing liquid(s) may be carried out by pumping or cascading under gravity.

The detergent station may comprise an automatic heater for regulating fluid temperature of the detergent station to be from about 28 degree Celsius to 38 degree Celsius. In tropical countries, water of room temperature (around 20˜28° C.) may be sufficient for dishwashing when using the present industrial dishwasher. In contrast, traditional industrial dishwashers have to involve hot water (i.e. 70° C. or above) or steam to remove greases from soiled serviceware. Advantageously, the present industrial dishwasher can utilise alkali electrolysis water and acid electrolysis water that are filled with tiny air cavities for providing effective washing to the serviceware. Energy consumption in heating the water is drastically reduced or eliminated. Factory floor of the industrial washer also becomes more user and environmental friendly. The present industrial dishwasher is thus known as low temperature dishwasher because the industrial dishwasher can wash dishes with water temperature at about 55˜65° C. or lower. For inflight catering centres, the industrial dishwasher can clean lightly soiled dishes with water temperature at 35˜45° C., or lower. Water of room temperature (20˜30° C.) may further be possible if washing time is slightly extended.

The sanitizing station can comprise a sanitizing container and a scouring container. The sanitizing container and the scouring container are contiguously or closely connected together for cascading used washing liquid from one container to the other. Used washing liquid from the sanitizing container is still useful for causing turbulence for scouring soiled serviceware in the scouring container. Accordingly, same amount of water is recycled for cost and energy saving.

The sanitizing station may comprises an Intelligent heater for keeping fluid temperature of the sanitizing station to be around 50 degree Celsius to 60 degree Celsius. Warm water (i.e. 45˜60° C.), but not hot water (i.e. 70° C. or above), in the sanitizing station enhances effectiveness of the sterilising agent.

The neutralisation station can comprise a prewashing container for mixing the cleaning agent with the sterilising agent. At the prewashing station, the cleaning agent, the sterilising agent, or both are neutralised, decomposed or degraded such that they will not harm environment when discharged. More importantly, used the cleaning agent, the sterilising agent or both are still useful to be stirred for running over soiled serviceware as scrubbing or brushing, preparing for subsequent washing.

The detergent station, the sanitising station, the neutralisation station or a combination of any of these stations may comprise one or more stirrers for causing turbulence in one or more of the stations. The stirrer may include one or more blades or pumps that agitates fluids in these stations such that the washing fluids can circulate around the serviceware for taking away food debris.

The industrial dishwasher can further comprise a rinsing station that is connected to the detergent station or the sanitising station for removing the cleaning agent, the sterilising agent or both cleansing agents. The rinsing station utilises turbulence of used washing fluid(s) for scrubbing the serviceware such that washing liquids from the detergent station and the sanitising station are recycled or reused for energy and cost saving.

The neutralisation station, the detergent station and the sanitising station may be serially connected for washing the serviceware from the neutralisation station, to the detergent station and then to the sanitising station sequentially. The serial connection provides convenience of washing liquids cascading from one station to the other under gravity, thus requiring no extra pumping. The serial connection is further suitable for deploying one conveyor chain that connects many washing stations together, as one integral industrial dishwasher.

The neutralisation station, the detergent station or the sanitising station can comprise one or more spray nozzles for discharging washing fluid onto the serviceware. Spraying of the washing liquid can be performed to both exposed and submerged serviceware. In fact, any washing stations of the industrial dishwasher can include one or more spray nozzles for effective fluid (gas or liquid) brushing, such as the rinsing station. The spray nozzle can be in the form of atomizer nozzle for producing fine droplets.

The industrial dishwasher may further comprise a drying station for parching the serviceware. Hot air, cold wind or Infrared light may be projected onto serviceware for removing water droplets effectively.

The Industrial dishwasher can further comprise a vision inspection station for examining washing quality of the serviceware automatically. The vision inspect station utilises machine vision or computer vision for examine surface condition of the serviceware either before or after dishwashing. Accordingly, the industrial dishwasher can select suitable programme, washing duration, alkaline level, acidity or cavity degree of the washing liquid(s) for effective washing. The vision inspection station can further reject and prevents unclean serviceware from being collected for future food packaging.

The industrial dishwasher may additionally comprise one or more steam stations for discharging steam onto soiled serviceware or washed serviceware. The steam station may be integrated with the drying station such that saturated or super-heated steam can remove water residue from washed serviceware. Particularly, stream of super-heated steam at high velocity can dry or sanitise washed serviceware, which is effective and consume less energy, as compared to traditional approach of heating up buckets of water in Industrial dishwashers continuously.

In the present application, the present industrial dishwasher can provide electrolysed water (i.e. alkali electrolysis water, acid electrolysis water or both) with ultrasonic wave for dish washing. The industrial dishwasher can further utilises electrolysed water with micro (i.e. cavity diameter of 10⁻³˜10⁻⁴ m) or ultra-fine cavities (i.e. cavity diameter of 10⁻⁸˜10⁻⁹ nm or smaller) for the dishwashing. The industrial dishwasher additionally can provide electrolysed water, cavities in the water and ultrasonic wave for the dishwashing. Besides, the industrial dishwasher can have electrolysed water, micro cavities (or ultra-fine cavities) and fluid (e.g. water) spraying for powerful washing. Various combination is applicable to be applied to different situation for cleaning in serviceware washing.

The industrial dishwasher may have one electrolyzer for producing both acid electrolysis water and alkali electrolysis water. Alternatively, the industrial dishwasher may have one generator for producing both alkali electrolysis water with cavities and acid electrolysis water with cavities. The industrial dishwasher may furthermore have one alkali electrolyser for producing alkali electrolysis water, and another acid generator for providing acid electrolysis water. Moreover, the industrial dishwasher may have one alkali cavitation generator for producing alkali electrolysis water with cavities, and another acid cavitation generator for providing acid electrolysis water with cavities.

Conventional industrial dishwashers typically need to operate at above 72 degree Celsius in order to sanitize the serviceware. A conventional industrial dishwasher typically heats up water at about 72 degree Celsius, which requires more than 20 KW for heating a small water tank. A lot of energy is used for heating water, which results in high operating cost and environmental harm. In contrast, the present industrial dishwasher can be configured to wash soiled dishes at low temperature, but yet achieve the hygiene quality for the washed dishes. In short, the resent industrial dishwasher washes soiled serviceware with water of low temperature (e.g. 45˜52° C.), but achieve better sanitizing. Since low temperature water requires less heating energy, the present industrial dishwasher consumes less electricity or gas for heating, thus saving money. Less heating of the industrial dishwasher may alternatively be known as more environmentally friendly or has less carbon footprint. Waste or used water from the industrial dishwasher is neutralized before disposal, thus causing less harm to the environmental. Particularly, since alkali electrolysis water and acid electrolysis water can neutralise each other, the present industrial dishwasher can cause little harm to the environment.

The present industrial dishwasher can utilise acid electrolysis water to spray, rinse, coat or wash serviceware for killing harmful bacteria or pathogen. Hence, the industrial dishwasher avoids the traditional high temperature washing (e.g. >72° C.), but achieve more effective sanitising result. The alkali electrolysis water and/or acid electrolysis water that are/is combined with tiny cavities can achieve excellent washing results, especially when further sprayed onto soiled serviceware via atomizer nozzles. The present industrial dishwasher reduces the need to rely on high temperature to loosen the grease/oil, the combination of tiny cavities in washing liquid(s), alkali electrolysis water, acid electrolysis water, spraying and ultrasonic washing has replaced the traditional high temp detergent spraying in conventional washer. In other words, all washing stations of the present industrial dishwasher deploys low temperature washing liquid(s) for cleaning soiled serviceware. The low temperature washing liquid(s) has temperature of about 16° C. to 70° C. at most or all stations. Preferably, the industrial dishwasher can use the one or more washing liquids at temperature of 22° C. to 56° C. If desired acidity and alkalinity levels of electrolysis water are provided, the industrial dishwasher can arrange all its washing stations (i.e. excluding drying station) to wash the soiled serviceware at 22° C. to 42° C., or even lower temperature.

According a second aspect of the invention, the present application provides a method for using an industrial dishwasher that comprises a step of providing a cleaning agent at a detergent station for washing serviceware; another step of offering a sterilising agent at a sanitising station for disinfecting the serviceware; and a further step of receiving and deactivating the cleaning agent, the sterilising agent or both at the neutralisation station for rinsing the serviceware. Some of these steps can be changed in sequence. For example, a user may offer the sterilising agent at the sanitising station before providing cleaning agent at a detergent station for washing serviceware. The neutralisation station is used to reduce potency of the cleaning agent, the sterilising agent or both such that the neutralisation station can discharge used washing liquid (e.g. dirty water) less harmful to the environment.

The method may further comprise a step of transporting soiled serviceware through, the neutralisation station, the detergent station and the sanitising station or a combination of any of these stations by one or more conveyor chains. Washing of serviceware can be sequentially or continuously performed by entering and leaving these stations such that the industrial dishwasher has high productivity and smaller environmental impact in terms of resource consumption (e.g. water).

The method can further comprise a step of discharging electric current through an electrolyte for making alkali electrolysis water as the cleaning agent, acid electrolysis water as the sterilising agent, or both simultaneously. Electrolysis is deployed by the industrial dishwasher for onsite or local production of electrolysed water, which includes alkali electrolysis water as detergent and acid electrolysis water as sanitizer. Storage time and transportation distance are minimised so that the electrolysed water can be used immediately after its production, providing its potency for usage at low cost.

The method may further comprise a step of generating micro cavities in washing fluid at the detergent station, the sanitising station, the neutralisation station or a combination of any of these stations. The micro or tiny cavities in the washing liquid can enhance washing capability and chemical stability of the electrolysed water. The micro cavities themselves are effective dislodging fat, food debris and stain from soiled serviceware. The micro cavities include cavities of diameters from 10⁻³ m to 10⁻⁶ m, and ultra-fine cavities of diameters from 10⁻⁶ m to 10⁻⁹ m or smaller. The micro cavities can carry negative charges at their surfaces and change fluid properties (e.g. viscosity, thermal conductivity, pressure dispersion) of liquid for better washing capability.

The method can further comprise a step of regulating fluid temperature of the detergent station, the sanitising station, the neutralisation station or a combination of any of these stations to be lower than 60 degree Celsius. Heat exchangers, heaters or chillers can be attached to or connected to any of these stations such that washing at these stations can be adjusted or optimised. In fact, washing liquid(s) of all washing stations (excluding drying station) of the present industrial dishwasher can be controlled at 60 degree Celsius or below for effective and speedy washing because electrolysed water (i.e. alkali electrolysis water and acid electrolysis water) and micro cavities in the electrolysed water can effectively wash soiled serviceware, having comparable cleaning results of traditional dishwashers with hot water (i.e. water hotter than 60 degree Celsius). Heating energy and monetary spending on surfactant are saved or reduced significantly. Therefore, the present industrial dishwasher may alternatively be known as low temperature dishwasher, green dishwasher or environmental friendly dishwasher.

According a third aspect of the present invention, the present application provides a method for making one or more industrial dishwashers. The method comprises a first step of providing a detergent station for washing serviceware with cleaning agent; a second step of offering a sanitising station for disinfecting the serviceware with sterilising agent; a third step of presenting a neutralisation station connected to the detergent station, the sanitising station or both for deactivating the cleaning agent, the sterilising agent or both at the neutralisation station, and a fourth step of giving one or more conveyor chains connected to the detergent station, the sanitising station or both stations for carrying the serviceware to the detergent station and the sanitising station. These stations can be modularly produced (individually manufactured) at factory, and can be flexibly assembled onsite for speedy installation. For maintenance or upgrading, some of these stations may be replaced or added with more stations for increasing washing capability of the industrial dishwashers.

The method can further comprise a step of connecting an electrolyzer to the detergent station and the sanitising station. The electrolyzer or electrolyser is a cost effective solution for producing both detergent and sanitising agent onsite, thus making the industrial dishwasher lost for long-term operation, because chemical detergent (e.g. surfactants) can be avoided.

The method further may comprise a step of joining a cavitation generator to the detergent station, the sanitising station, the neutralisation station, the electrolyzer or a combination of any of these. The cavitation generator provide micro or ultra-fine cavities in washing liquids (e.g. water) such that the micro or ultra-fine cavities are both effective in dislodging food debris, and enhancing performance of the detergent.

According to a fourth aspect, an industrial dishwasher of the present application comprises a washing station for cleaning dishes with water and an electrolyser connected to the washing station for providing electrolysed water to the washing station.

The electrolyser that produces electrolysed water onsite. Alkaline water solution near a cathode of the electrolyser is used as detergent, whilst acidic water solution near an anode of the electrolyser is employed as sanitizer. Used alkaline water solution and acidic solution can be mixed for neutralisation such that they can be safely discharged to sewer or ordinary drains.

The alkaline water solution serves as detergent for removing oily, protein, fats and stain on serviceware items (e.g. dishes), whilst the acid water solution is used as sanitizer for removing, germs, bacteria and virus. A detergent wash tank of the industrial dishwasher is filled with the alkaline water solution for direct spraying into the detergent wash tank (e.g. bucket compartment) to wash the serviceware items. Thereafter, the serviceware items are transported to a sanitizing washing zone such that a sanitizing tank of the sanitizing washing zone is filled with the acidic water solution for direct spraying onto the serviceware items in the sanitizing tank. Used alkaline water solution will then be directed to a first power wash tank for recycling by flushing dirty wares (soiled dishes) as the dirty wares enter the first power wash tank (washer). In contrast, acidic water solution is directed to a second power rinse tank for recycling in order to rinse out debris of serviceware items. Both used/exhausted alkaline and acid water solutions are finally discharged via a common drain mixer in order to neutralise the waste water thus achieving zero impact to the environment.

The electrolyser has one or more pH meters that measure pH (acidity or alkalinity) of a liquid (e.g. water solution). The one or more pH meters have sensor(s) or measuring probe(s), which are glass electrodes connected to electronic meter(s) for measuring and displaying pH readings or values. Production rates of the electrolyser are regulated according to measured values at one or more washing stations of the industrial dishwasher. For example, the electrolyser adjusts its output rate of water solution for alkaline water solution for maintaining ph8.3 or higher at a (power) prewash washing station such that grease on soiled dishes can be effectively and efficiently dissolved. In the contrast, the electrolyser regulates its export flow rates of its acidic water solution for maintaining pH6.3 or lower at a sanitising washing station such that germs on dishes may be effectively and efficiently exterminated.

The industrial dishwasher provides its own detergent and sanitizer onsite such that it requires less or none external supply of detergents and sanitizers. Since typical commercially available detergents and sanitizers are chemical solutions, the industrial dishwasher has less environmental impact, causing less or none pollution when washing dishes.

The industrial dishwasher may include an ultrasonic generator having a push type ultrasonic transducer. The push type ultrasonic transducer satisfies demanding cleaning and sonochemistry applications, especially for food industries. The push type ultrasonic transducer has solid titanium alloy radiator that provides highest durability. The push type ultrasonic transducer further provides 360 degree radiating field providing omndirectional energy and minimum dead-spot area. The transducer also has high efficiency, exceeding 95%. The transducer is particularly suitable for cleaning under vacuum or high pressure, sonoradiation of reagents and general sonochemistry. When used with corresponding generators, the transducer is safe under dry-run conditions, has small footprint, simple retrofit to existing tank systems, and is suitable in 20, 25, 30 and 40 kHz operating frequencies. The transducer is submersible for radiating energy omni-directionally. Standing waves of the transducer are thus less likely to develop and uniform activity within a volume of fluid is attainable at efficiency better than 95%.

The industrial dishwasher avoids using hot water for removing grease or killing germs, known as cool wash. For example, known dishwashers often use hot water (e.g. 72° C.) for dissolving oily food debris or sanitising dishes. In contrast, the present industrial dishwasher maintains water temperature in some or all of the washing stations to be 50° C. or lower. Energy consumption for water heating is greatly reduced or eliminated. Instead, the industrial dishwasher deploys electrolysed water and ultrasonic waves to clean soiled dishes effectively, which further has feedback loop(s) for achieving predetermined pH values at designated washing stations. In tropical environment, the industrial dishwasher can circulate water of 30° C. for dishwashing without heating the water at all. In cold environment, the industrial dishwasher only needs to moderately warm up the water for washing at 20˜30° C. In other words, the present dishwasher does not require raise temperature of the water for dissolving grease or killing germs.

Following the fourth aspect, the industrial dishwasher comprises a washing station for cleaning dishes with water, an ultrasonic generator attached to the washing station for agitating the water with ultrasonic wave, an electrolyser connected to the washing station for providing electrolysed water to the washing station, and wherein the ultrasonic generator further comprises an ultrasonic controller or sensor for agitating the water with ultrasonic waves of various frequencies.

The ultrasonic generator can comprise one or more ultrasonic transducers for generating the various frequencies respectively. The one or more ultrasonic transducers may be mounted at on a bottom side of an ultrasonic cleaning tank. The one or more ultrasonic transducers can comprise a piezoelectric transducer, a magnetostrictive transducer or both. The ultrasonic controller may further comprise a sweep frequency circuit that is connected to one or more of the plurality of ultrasonic transducers for generating the ultrasonic waves of various frequencies.

The electrolyser can comprise an anode for providing acidic water (anolyte) to an acid washing station and a cathode for giving alkaline water (Catholyte) to an alkaline washing station (separated from the acidic water), (the anode and the cathode being optionally separated by a porous membrane). The acidic washing station and the alkaline washing station may be sequentially connected with or without other washing stations in-between.

The industrial dishwasher can further comprise a neutralising station for neutralising the acidic water and the alkaline water before draining. The Industrial dishwasher may comprises one or more rinsing station connected before or after to one of the acidic washing station, the alkaline washing station or the neutralising station. The washing station can comprise a scrap washing station, a prewashing station, a detergent washing station, a rinse washing station, a sanitizing washing station, a rinse washing station or a combination of any of these washing stations. Two or more of the scrap washing station, a prewashing station, a detergent washing station, a rinse washing station, a sanitizing washing station and a final rinse washing station may be contiguously or Indirectly connected together such that the water can flow from one of the two washing stations to the other.

The prewashing station and the rinse washing station can be connected together such that water from rinse washing station can flow to the prewashing station. The rinse washing station and the final rinse washing station may be connected together such that water from final rinse washing station can flow to the rinse washing station. The scrap washing station, the prewashing station, the detergent washing station, the rinse washing station, the sanitizing washing station, the rinse washing station or a combination of any of these washing stations may comprise the ultrasonic generator.

The scrap washing station, the prewashing station, the detergent washing station, the rinse washing station, the sanitizing washing station, the rinse washing station or a combination of any of these washing stations can further comprise an electrode for generating electrolysed water locally. The washing station may comprise a stirrer for causing water flow inside the washing station.

The washing station further can comprise a grille at a side for preventing contact between washing the dishes and parts of the washing station. The washing station may additionally comprise a heater with a thermostat for controlling cleaning solution in the washing station. The washing station can further comprises one or more ISFET pH electrode or pH sensor for checking pH value of water solution in the washing station.

The electrolyser may be connected to the one or more ISFET pH electrode or pH sensor for regulating output rates of electrolysed water depending on pH values at the washing station. The ultrasonic generator can comprise a push type ultrasonic transducer. The sanitizing washing station may comprise a temperature regulator or heater for maintaining water temperature from 40 to 75 degree Celsius. All washing stations may be configured for washing dishes at room temperature.

The accompanying figure (FIG.) illustrates embodiments and serves to explain principles of the disclosed embodiments. It is to be understood, however, that these figures are presented for purposes of illustration only, and not for defining limits of relevant inventions. In particular,

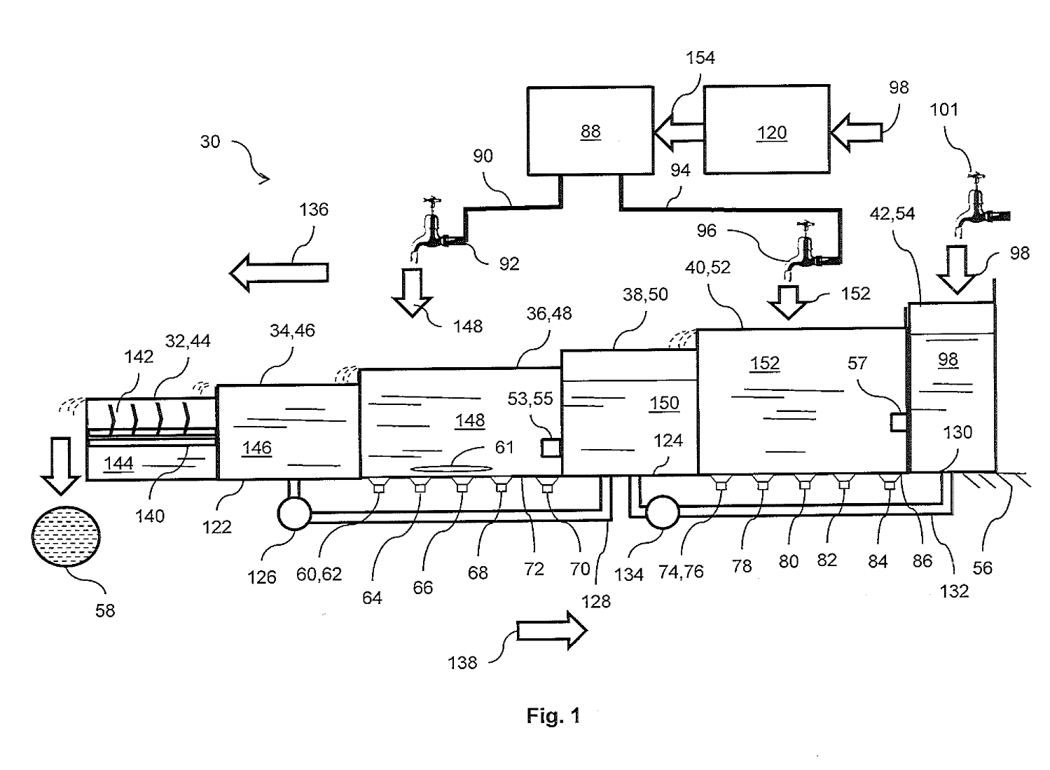

FIG. 1 illustrates a schematic diagram of a first industrial dishwasher 30, whilst

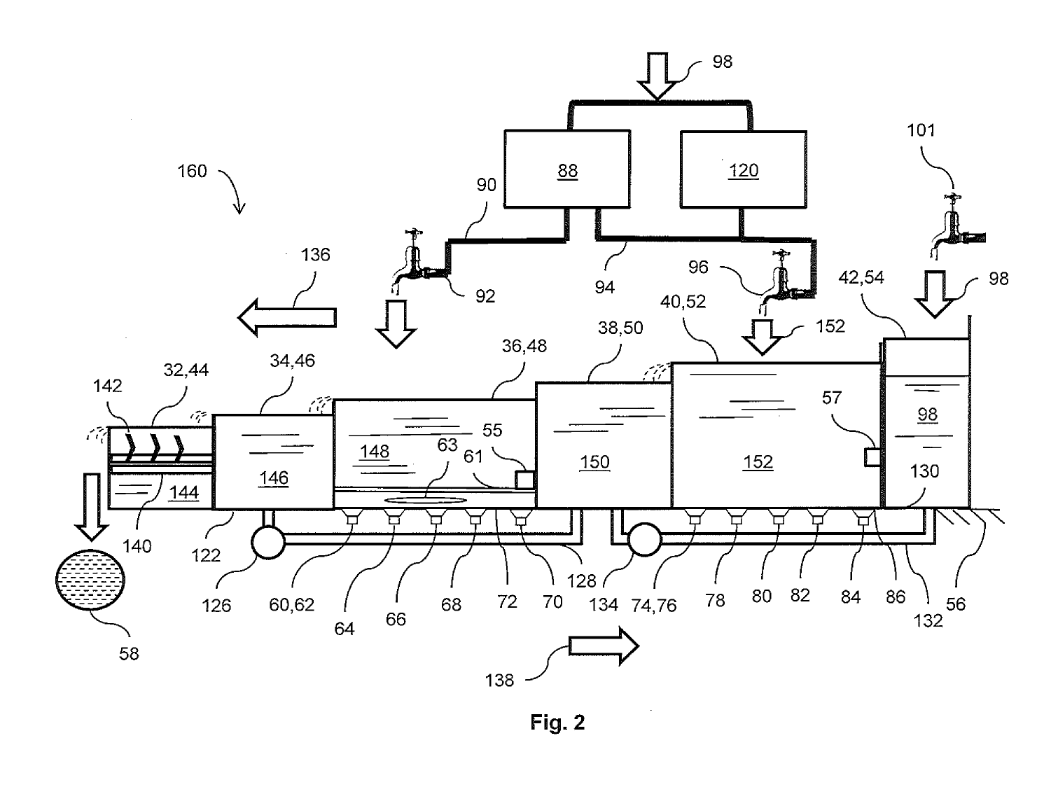

FIG. 2 illustrates a simplified graph of a second industrial dishwasher 160.

Exemplary, non-limiting embodiments of the present application will now be described with references to the above-mentioned figures. FIG. 1 relates to an embodiment of the present application, which is the first industrial dishwasher 30. The first industrial dishwasher 30 comprises a scraping station 32, a prewashing station 34, a detergent station 36, a scouring station 38, a sanitizing station 40 and a rinsing station 42 that are sequentially and contiguously connected next to each other. Accordingly, these respective washing stations 32-42 are alternatively known as a first washing station 32, a second washing station 34, a third washing station 36, a fourth washing station 38, a fifth washing station 40 and a sixth washing station 42. Each of these washing stations 32-42 has a container 44-54 whose bottom (i.e. bottom side) is supported by a factory floor 56 of the same level. These containers 44-54 consist of a scraping container 44, a prewashing container 46, a detergent container 48, a scouring container 50, a sanitizing container 52 and a rinsing container 54 for each of these stations 32-42 respectively. Similarly, these containers 44-54 are alternatively known as a first container 44, a second container 46, a third container 48, a fourth container 50, a fifth container 52 and a sixth container 54.

Heights of these containers 44-54 increase progressively from the first container 44 to the sixth container 54. In other words, the first container 44 is shorter than the second container 46; the second container 46 is shorter than the third container 48; the third container 48 is lower than the fourth container 50; the fourth container 50 has a smaller height than the fifth container 52; and the fifth container 52 is lower than the sixth container 54.

The detergent station 36 further includes an automatic heater 55 having an inbuilt thermostat 53. The automatic heater 55 is configured to turn on the automatic heater at 54° C. and switch off the automatic heater at 60° C. such that water inside the detergent container 48 is kept around 57° C. Similarly, the sanitising station 40 further has an intelligent heater 57 with an integrated thermostat 59. Water inside the fifth container 52 is regulated at about 30±5° C. by the Integrated thermostat 59 for preventing protein coagulation.

The first industrial dishwasher 30 further comprises an electrolyzer 88 and a fine cavity generator 120 that are serially connected to a clean water tap 101. The fine cavity generator 120 has a cylinder for guiding high-speed rotational flow of water, which further produces a gas, gas-liquid and a liquid layer. Friction between these three layers generates nanoscopic sized cavities in the water called fine cavities. These fine bubbles have low buoyancy and remain suspended in the water for a long period of time. The air-cavity saturated water 154 is oxygen-rich, whilst surfaces of the find air cavities are (electrically) negatively charged. Accordingly, the fine air cavities attract and attach themselves to organic materials and give the water 154 stronger rinsing effect. The water and air mixture 154 with an oxidative gas (e.g. dissolved air, oxygen or ozone) can effectively disinfectant and reduce both chemical and clean water consumption. When required, the water and air mixture 154 can further be injected and dissolve with carbon dioxide for efficiently neutralizes alkalinity in liquids. Diameter of the micro air cavities are about from one-billionth of a meter to about one millionth of a meter. The micro air cavities can enter deep into surface imperfections (e.g. crevices and cracks) such that they can dig out and remove dirt and odours, leaving clean surfaces on dishes.

The fine cavity generator 120 is connected to the tap for receiving clean water, whilst the electrolyzer 88 is further connected to the fine cavity generator 120 at its downstream. The electrolyzer 88 has an alkaline water pipe 90 and an acid water pipe 94. Ends of these two pipes 90, 94 are installed with an alkaline valve 92 and an acid valve 96 respectively. The alkaline valve 92 is connected to the third container 48, whilst the acid valve 96 is connected to the fifth container 52 respectively.

The final rinse washing station 42 is connected to the tap 101 such that the sixth container 54 is configured to receive clean water from the tap 101 when washing dishes. In contrast, the scrap washing station 32 is connected to sewer 58 such that overflowing water from the first container 44 drains to the sewer 58.

A bottom side 122 of the prewash station 34 is connected to a bottom side 124 of the scouring station 38 via a pump 126 and a tube 128 such that water can discharge from the scouring station 38 to the prewash station 34 directly, without flowing to the detergent station 38. Similarly, a bottom side 130 of the rinsing station 42 is again connected to the bottom side 124 of the scouring station 38 via a tube 132 and an Inline pump 134 such that water of the rinsing station 42 can flow immediately to the scouring station 38, instead of running from the sixth container 54, to the fifth container 52 and to the fourth container 50. The sanitising station 40 and the detergent station 36 have access to external incoming water through valves 92, 96 respectively.

The detergent station 36 has an ultrasonic generator 60 that comprises a first ultrasonic transducer 62, a second ultrasonic transducer 64, a third ultrasonic transducer 66, a fourth ultrasonic transducer 68 and a fifth ultrasonic transducer 70. These ultrasonic transducers 62-70 are evenly distributed and attached to a bottom 72 of the third container 48. Active elements of these ultrasonic transducers 62-70 include rings of lead zirconate titanate, which are bolted to aluminium coupling horns respectively. The ultrasonic generator 60 can produce ultrasonic waves of 20˜40 kHz when powered. The detergent station 36 also has a grille 61 near, but above the bottom 72 of the third container 48. A stirrer 63 is installed between the grille 61 and the bottom 72. The stirrer 63 has blades (now shown) that can circulate water inside the third container 48.

The detergent station 36 utilise alkaline clean water 148 that is also saturated with micro sized air cavities. The alkaline clean water 148 is powerful for cleaning, disinfecting and deodorizing because the alkaline clean water 148 can effectively break up oils and proteins, lifting oxidized matter away from surfaces to clean tough stains. The fine air cavities can penetrate into microscopic cracks and crevices below the surface, thereby pulling stains, dirt, and bacteria out of these microscopic cracks and crevices. The fine air cavities drive electrolyzed water deep into surfaces of the dishes for having greater disinfecting and cleaning effect. The fine air cavities can further keep effective components of the electrolyzed water 148 to be more stable, such as for several weeks.

Similarly, the sanitising washing station 40 has another ultrasonic generator 74 that has a sixth ultrasonic transducer 76, a seventh ultrasonic transducer 78, an eighth ultrasonic transducer 80, a ninth ultrasonic transducer 82 and a tenth ultrasonic transducer 84. These ultrasonic transducers 76-84 are evenly distributed and attached to a bottom 86 of the fifth container 52.

FIG. 1 further depicts a conveyor chain 140 and soiled serviceware (e.g. plates or dishes) 142 at the scarping station 32. The conveyor chain 140 and the soiled serviceware 142 are extended into the remaining stations 34, 36, 38, 40, 42 too, but are omitted for clarity and ease of illustration. The serviceware 142 are substantially submerged under washing liquids 144, 146, 148, 150, 152, 98 of these stations 32, 34, 36, 38, 40, 42.

The electrolyzer 88 can generate electrolysed water (“Electrolyzed Water”, EOW or ECA), which is also known as electrolyzed oxidizing water, electro-activated water or electro-chemically activated water solution. The electrolyzer 88 produces the electrolysed water by the electrolysis of ordinary tap water containing dissolved sodium chloride (NaCl). Typically, tap water has sufficient dissolved salts for normal operation of the electrolyzer 88. The electrolysis of such salt solutions produces a solution of sodium hypochlorite, which is the most common ingredient in store-bought household bleach. The resulting water is a known cleanser and disinfectant/sanitizer, but not a surfactant (soap). The electrolyzer 88 also produces acidic electrolyzed water for disinfecting and deodorising.

In contrast, the fine cavity generator 120 can generate tiny air pockets in solvents (e.g. water), which typically less than 1 mm in diameter. In the fine cavity generator 120, air and water flow tangentially into a cylinder which generates a high-speed rotational flow for separating them into an air, air-water and water layer. Friction between the swirling layers creates the tiny air pockets which are suspended in the water for a prolonged period of time, in contrast with air bubbles of more than 1 mm in diameter. According to Brownian motion particle theory, these tiny air pockets will randomly drift and remain in water without being affected by buoyancy. In practice, air pockets with diameter of up to about one-billionth of a meter (i.e. 10⁻⁶˜10⁻⁹ m). These suspended air pockets in water has diameters in the micrometre size (10⁻³ meters) or smaller are called fine air pockets. Nanometre-sized air pockets (between 100 and 300 nm) are called ultra-fine air pockets. Here, air pockets are also known as air bubbles or simply bubble.

When is use, fresh tap water 98 is poured into the sixth container 54 by the tap 101 at a flow rate of 32 litres per minute. The fresh tap water 98 fills the sixth container 54 and is circulated within the rinsing container 54 by impellers (not shown). The clean water 98 is further propelled by the pump 134 from a bottom 130 of the sixth container 54 to the fourth container 50 at its bottom 124. Water 150 of the fourth container 50 is further impelled by the other pump 126 from its bottom 124 to the second container 46 at the bottom 122.

When pouring the fresh clean water 98 into rinsing station 42, portion of the clean water 98 also enters the fine cavity generator 120, which generates micro-sized air cavities saturating the clean water. The water and air mixture 154 further progresses into the electrolyzer 88 for undergoing electrolysis process. Water of about pH11˜12 with micro cavities 148 is discharged from the alkaline or alkali valve 92 for entering the detergent station 36, whilst water of pH about 3.5 with micro cavities 152 is released to the sanitizing station 40 via the acidic or acid valve 92.

Simultaneously, excess water 152 from the sanitising station 40 overflows from the fifth container 52 to the fourth container 50, similar to the form of waterfall or cascade. Superfluous water 148 from the detergent station 36 further pours from a rim of the third container 48 to the second container 46. Surplus water 146 of the prewashing station 34 overspills from an edge of the second container 46 to the first container 44 of the scarping station 32 under gravity. Runoff water 144 of the scraping station 32 floods the first container 44 such that runoff water 144 is discharged to the sewer 58 below. Accordingly, clean water 98 generally cascades down from the rinsing station 42 to the scraping station 32 during dishwashing operation, known water flow direction 136. In contrast, service ware items (not shown) are carried on a conveyor chain (not shown), moving from the scarping station 32 to the rinsing station 42, known as progress or production direction 138. The service ware, serviceware or serviceware items include food utensils, trays and tableware items (e.g. dishes or dishware, cutlery) that are used for food serving.

During dishwashing process, soiled dishes or tableware items (not shown) are initially placed onto the conveyor chain and dipped into (i.e. full immersed) the scraping station 32. Food debris is flushed off from those soiled or smeared when the soiled dishes are immersed in the first container 44 under turbulent streams (not shown). Although water in the scraping container 44 is generally dirty and neutralised, the neutralised dirty water 144 is swirled inside the scraping container 44 for scratching food debris off surfaces of the soiled dishes. After a predetermined period of time (e.g. about 3 minutes), the soiled dishes are progressively carried out of the scraping container 44 by the conveyor chain and further enter the prewashing station 34. Particularly, waste water 144 that runs off from the scarping container 44 is drained into a sewer 58.

The soiled dishes are also full submerged in the prewashing (second or 2^{nd}) container 46. The second container 46 holds mixed water 146 of lower acid concentration level. Wild water current in the prewashing container 46 is tossed over the soiled dishes again such that majority of visible food debris or oil/cream is removed from those soiled dishes. The prewashing station 34 utilises a vacuum pump (not shown) to pull the washing liquid (mixed water) 146 in and push the washing liquid 146 back out of jets (not shown) on walls of the prewashing container 46. The prewashing station 34 has an Intake valve (not shown) at bottom 122 with a fine mesh screen covering (not shown). The mixed water 146 is drawn in with a vacuum created by a pump motor (not shown). The pump motor pulls the mixed water 146 in and expels it from the jets placed throughout the prewashing container 46. After a predetermined period of time (e.g. about 2.4 minutes), the conveyor chain further carries the soiled dishes into the detergent station 36.

The third container 48 of the detergent washing station 36 that receives alkaline clean water 148 from the alkaline valve 92, and the alkaline clean water 148 is produced from a cathode portion/compartment (not shown) of the electrolyser 88. The alkaline clean water 148 from the cathode portion (cathodic solution) has hydrogen gas and hydroxide ions, leading to an alkaline solution that has essentially sodium hydroxide. In contrast, at an anode portion/compartment (not shown) of the electrolyser 88, chloride ions are oxidised to elemental chlorine. Some of the elemental chloride is combined with some of the hydroxide ions produced from the cathode portion/compartment, where the water/solution disproportionates into hydrochlorous acid, a weak acid and an oxidising agent. This “acidic electrolyzed water” Is further raised to pH2.7˜5.0 or higher (i.e. with lower pH value) by mixing in the desired amount of hydroxide ion solution from the cathode compartment, yielding a solution of sodium hypochlorite NaClO which is the major component of ordinary household laundry bleach. The solution contains equal concentrations of hypochlorous acid and hypochlorite ion. In other words, the soiled dishes are washed by locally (on-site) produced detergent made from electrolysis. The alkaline clean water 148 is saturated with air cavities such that dirt and grease are effectively removed from the soiled dishes.

At the bottom 72 of the third container 48, the ultrasonic generator 60 is energised such that the first ultrasonic transducer 62 produces ultrasonic standing wave at 20 kHz, the second ultrasonic transducer 64 emits ultrasonic standing wave at 100 kHz, the third ultrasonic transducer 66 releases ultrasonic standing wave at 180 kHz, the fourth ultrasonic transducer 68 discharges ultrasonic standing wave at 260 kHz, whilst the fifth ultrasonic transducer 70 emancipates ultrasonic standing wave at 400 kHz. The ultrasonic generator 60 has a sweep frequency circuit (not shown) that is connect to these ultrasonic transducers 62-70. The sweep frequency circuit causes these ultrasonic transducers 62-70 to produce ultrasonic waves within a range of ±10 kHz of the predetermined standing wave frequencies. Accordingly, the detergent washing station 36 deploys ultrasonic sweep cleaning technique that “sweeps” multiple ultrasonic frequencies through the single detergent container 48. This ultrasonic sweep cleaning technique controls creation of standing waves, allowing no single frequency to resonate in the tank, eliminating the root cause of standing waves, and tank dead spots. In the meantime, the stirrer 61 rotates its blades such that water solution 148 inside the third container 48 is swirled over the dishes. The heater 55 provides heat to the water solution and maintains the water solution at about 54±5° C. for effective cleaning.

The conveyor chain transports the soiled dishes further into the fourth container 38, where the soiled dishes experience water current of spiral forms around six opposite directions according to Cartesian coordinate system (not shown). Each direction of the rinsing current is independently regulated by its flow rate, intensity and pressure. The scouring container 50 receives water from both the sanitizing container 52 by cascading and the rinsing container 54 via the pump 134. Hence, water 150 inside the souring container 50 has high pH value (less acidic) as compared to that of the sanitizing station 40. After the scouring station 38, the soiled dishes are generally free from visible food debris or grease.

Semi-washed dishes (not shown) are shipped by the conveyor chain to the sanitizing station 40 successively. The ultrasonic generator 74 at bottom 86 of the sanitizing station 40 is switched on such that the sixth ultrasonic transducer 76 produces ultrasonic standing wave at 50 k Hz, the seventh ultrasonic transducer 78 emits ultrasonic standing wave at 130 k Hz, the eighth ultrasonic transducer 80 releases ultrasonic standing wave at 210 k Hz, the ninth ultrasonic transducer 82 discharges ultrasonic standing wave at 290 k Hz and the tenth ultrasonic transducer 84 emancipates ultrasonic standing wave at 370 k Hz. The ultrasonic generator 74 has a sweep frequency circuit (not shown) that is connect to these ultrasonic transducers 76-84. The sweep frequency circuit causes these ultrasonic transducers 76-84 to produce ultrasonic waves within a range of ±15 kHz of the predetermined standing wave frequencies. Accordingly, the sanitizing station 40 deploys ultrasonic sweep cleaning technique that “sweeps” multiple ultrasonic frequencies through the single sanitizing container 52. This ultrasonic sweep cleaning technique controls creation of standing waves, allowing no single frequency to resonate in the tank, eliminating the root cause of standing waves, and tank dead spots. In the meantime, the intelligent heater 57 heats up the water solution for maintaining the water solution 152 at about 55±5° C. for effective sanitising.

The water solution 152 in the sanitizing container is highly acidic because the sanitizing station 40 receives clean acidic water 152 (or acidic clean water) directly from the acidic valve 96. In contrast to the water solution from the cathode-portion used as mild all-purpose detergent, the water solution from the anode-portion acts as a food-grade sanitizer that is highly effective as a virucide, bacteriacide, and sporacide. The acidic clean water 152 is additionally full of micro-sized air cavities whose diameters are about 10⁻⁶˜10⁻⁹ meter. These air cavities are suspended in water with prolonged period of time such that the air cavities enter crevice of the soiled dishes.

The conveyor chain additionally brings washed dishes from the sanitising station 40 to the rinsing station 42. These dishes are flushed with powerful water current in the sixth container 54 such that they are almost free from any grease, food debris, germs and bacteria, suitable for contacting edible food items.

The industrial dishwasher 30 has a drying station (not shown) that is joined to the rinsing station 42. The drying station produces air-knife or air-blade (narrow strip of airflow at high speed) (not shown) that pass over cleaned dishes. The drying station also has hot air blowers that propel hot air at about 65˜95° C. over the cleaned dishes. The drying station further has fans (not shown) that circulate ambient air over the cleaned dishes for drying. Cleaned and dried dishes are collected and collated at an end of the conveyor chain.

A vision inspection station (not shown) of the industrial dishwasher is positioned next to the drying station. The vision inspection station has cameras and industrial robots (also known as robotic arm) whereby the industrial robots can grasp dried dishes in front of the cameras respectively for examining surface integrity of the dishes. If water marks, streaks, dirt or cracks are found on the dishes, the dishes are placed into a reject bin for re-washing at the scraping station or disposal. Hence, only clean and good dishes are accepted, sorted and stacked into baskets for food packaging.

In the embodiment, the first ultrasonic transducer has an insertion of a resonance enhancing disc made of alumina ceramic, which is positioned between a base of ultrasonic transducer and a piezo electric material. This structure provides an Increase in intensity of the resonant frequency signals, diminishing periodical shift in frequency and stabilizing piezo electric material temperature. In other words, the ultrasonic generator 60 is a megasonic processing apparatus that comprise one or more piezoelectric transducers 62-70. The first ultrasonic transducer has a fundamental resonant frequency of at least 300 KHz; a tank adapted to contain fluid and one or more parts to be processed. One or more of the transducers are adapted for providing vibrations to the tank and its contents; a generator coupled to the transducers for supplying a driving signal at a variable frequency throughout a frequency range that includes the resonant frequencies of all the transducers.

The transducers 62-70, 76-84 are arranged in equilateral triangular patterns along diagonal lines on a wall of the tanks respectively 48, 52 so that each transducer 62-70, 76-84 has an adjacent transducer 62-70, 76-84 of a different frequency. Alternatively, the apparatus includes one or more rod transducers having different resonant frequencies so that the apparatus provides a mixture of various frequencies of ultrasonic energy to the tank. The embodiment involves selecting transducers with different resonant frequencies that are outside an excluded subrange, and powering the transducers by a driving signal that sweeps through the resonant frequencies of the transducers and the excluded subrange. In other words, the industrial dishwasher comprises one or more tanks/containers 48, 54 operable for containing a fluid; multiple ultrasonic transducers 62-70, 76-84 coupled to the tanks 48, 52 and operable for supplying ultrasonic energy to the fluid/water in the tanks 48, 52. A first group of the transducers 62-70, 76-84 have a first resonant frequency and a second group of the transducers 62-70, 76-84 have a second resonant frequency that is different from the first resonant frequency. The transducers 62-70, 76-84 are arranged in an equilateral triangular pattern along diagonal lines so that each transducer 62-70, 76-84 has at least two adjacent transducers 62-70, 76-84 and at least one adjacent transducer 62-70, 76-84 has a different resonant frequency; and a generator means for supplying driving signals to the transducers 62-70, 76-84.

The ultrasonic generators 60, 74 are megasonic processing apparatuses having one or more piezoelectric transducers 62-70, 76-84 operating in thickness mode at fundamental resonant frequencies of at least 300 kHz. The ultrasonic generators 60, 74 powers the transducers 62-70, 76-84 with a variable-frequency driving signal that varies or sweeps throughout a predetermined sweep frequency range. The ultrasonic generators 60, 74 repeatedly vanes or sweeps the frequency of the driving signal through a sweep frequency range that includes the resonant frequencies of all the transducers 62-70, 76-84. In other words, the ultrasonic generators 60, 74 are megasonic processing apparatuses that comprise one or more piezoelectric transducers, each having a fundamental resonant frequency of at least 300 KHz. Tanks 48, 52 or containers of the industrial washer 30 adapted to contain fluid and one or more parts to be processed. The ultrasonic transducers 62-70, 76-84 are adapted for providing vibrations to the tanks 48, 52 and its contents (soiled dishes). One or more generators are coupled to the transducers 62-70, 76-84 for supplying a driving signal at a variable frequency throughout a frequency range that includes the resonant frequencies of all the transducers 62-70, 76-84.

FIG. 2 illustrates a simplified graph of a second industrial dishwasher 160. The second embodiment comprises parts or method steps that are similar or identical to those of the first industrial dishwasher 30. These similar or identical parts of method steps are thus labelled with similar or identical reference numerals. Description of these relevant parts of method steps is hereby incorporated by reference, wherever relevant or appropriate.

Particularly, the second industrial dishwasher 160 comprises an electrolyzer 88 and a fine cavity generator 120 that are connected in parallel. According to FIG. 2, the electrolyzer 88 is connected to the tap 101 directly for receiving clean water 98 and is further connected to the alkaline valve 92 for discharging to a detergent station 36. Similarly, a fine cavity generator 120 is also joined to the tap 101 straight for receiving the clean water 98 and is further linked to an acid valve 96 for pouring into a sanitizing station 36. In addition, the second pipe 94 joins the electrolyzer 88 to the third pipe 162 of the fine cavity generator 120 such that acidic clean water 152 from the electrolyzer 88 mixes with clean water saturated with fine water cavities 154 for delivery into the sanitizing container 52.

When using the second industrial dishwasher 160, the detergent station 36 deploys highly alkaline water for removing grease and food residue, whilst the sanitizing station 40 makes use of both air cavity saturated water 154 and acidic clean water 152 for sterilising serviceware items.

In the application, unless specified otherwise, the terms “comprising”, “comprise”, and grammatical variants thereof, intended to represent “open” or “Inclusive” language such that they include recited elements but also permit inclusion of additional, non-explicitly recited elements.

As used herein, the term “about”, in the context of concentrations of components of the formulations, typically means+/−5% of the stated value, more typically+/−4% of the stated value, more typically+/−3% of the stated value, more typically, +/−2% of the stated value, even more typically+/−1% of the stated value, and even more typically+/−0.5% of the stated value.

Throughout this disclosure, certain embodiments may be disclosed in a range format. The description in range format is merely for convenience and brevity and should not be construed as an Inflexible limitation on the scope of the disclosed ranges. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

It will be apparent that various other modifications and adaptations of the application will be apparent to the person skilled in the art after reading the foregoing disclosure without departing from the spirit and scope of the application and it is intended that all such modifications and adaptations come within the scope of the appended claims.

### REFERENCE NUMERALS

30 first industrial dishwasher
32 scraping station
34 prewashing station
36 detergent station
38 scouring station
40 sanitizing station
42 rinsing station
44 scraping container
46 prewashing container
48 detergent container
50 scouring container
52 sanitizing container
53 inbuilt thermostat
54 rinsing container
55 automatic heater
56 factory floor
57 intelligent heater
58 sewer
59 integrated thermostat
60 first ultrasonic generator
61 grille
62 first ultrasonic transducer
64 second ultrasonic transducer
66 third ultrasonic transducer
68 fourth ultrasonic transducer
70 fifth ultrasonic transducer
72 bottom
74 second ultrasonic generator
76 sixth ultrasonic transducer
78 seventh ultrasonic transducer
80 eighth ultrasonic transducer
82 ninth ultrasonic transducer
84 tenth ultrasonic transducer
86 bottom
88 electrolyzer (Alkaline water electrolysis)
90 alkaline pipe
92 alkaline valve
94 acid pipe
96 acid valve
98 fresh tap water
101 tap
120 fine cavity generator
122 bottom side
124 bottom side
126 pump
128 tube
130 bottom side
132 bottom side
134 pump
136 water flow direction
138 progress direction
140 conveyor chain
142 serviceware
144 neutralised dirty water
146 mixed water
148 alkaline clean water
150 acidic used water
152 acidic clean water
154 water and air mixture
160 second industrial dishwasher
162 third pipe

1. An industrial dishwasher, comprising
a detergent station for washing serviceware with a cleaning agent;
a sanitising station for disinfecting the serviceware with a sterilising agent;
a neutralisation station connected to at least one of the detergent station, and the sanitising station for deactivating at least one of the cleaning agent and the sterilising agent; and
at least one conveyor chain connected to at least one of the detergent station, and the sanitising station for carrying the serviceware to the other of the detergent station and the sanitising station.

2. The industrial dishwasher of claim 1, wherein
the at least one conveyor chain is further connected to the neutralisation station for rinsing the serviceware at the neutralisation station.

3. The industrial dishwasher of claim 1,
wherein the at least conveyor chain comprises an endless belt for moving the serviceware from the detergent station to the sanitising station or vice versa.

4. The industrial dishwasher of claim 1 further comprising
an electrolyzer for at least one of: supplying the cleaning agent to the detergent station, the sterilising agent to the sanitising station, and both the cleaning agent to the detergent station and the sterilising agent to the sanitising station.

5. The industrial dishwasher of claim 4, wherein
the electrolyzer is configured to produce at least one of alkali electrolysis water as the cleaning agent and acid electrolysis water as the sterilising agent.

6. The industrial dishwasher of claim 1 further comprising
a cavitation generator connected to the detergent station, the sanitising station, the neutralisation station or a combination of any of these stations for producing voids in at least one washing liquid.

7. The industrial dishwasher of claim 6, wherein
the cavitation generator comprises a mixer for injecting the gas into the at least one washing liquid to form micro or ultra-fine cavities.

8. The industrial dishwasher of claim 7, wherein
the mixer comprises a porous cylinder for guiding the gas and the liquid flowing along opposite sides of the porous cylinder.

9. The industrial dishwasher of claim 6, wherein
the cavitation generator comprises an ultrasonic generator.

10. The industrial dishwasher of claim 9, wherein
the ultrasonic generator comprises at least one ultrasonic transducer on a wall of at least one washing station.

11. The industrial dishwasher of claim 10, wherein
the ultrasonic generator comprises multiple ultrasonic transducers evenly distributed on a bottom side of the at least one washing station.

12. The industrial dishwasher of claim 1, wherein
the detergent station comprises a detergent container and a prewashing container, the detergent container and the prewashing container connected together for cascading used at least one washing liquid from one container to another container.

13. The industrial dishwasher of claim 1, wherein
the detergent station comprises an automatic heater for regulating fluid temperature of the detergent station to be from about 28° C. to about 38° C.

14. The industrial dishwasher of claim 1, wherein
the sanitizing station comprises a sanitizing container and a scouring container, the sanitizing container and the scouring container connected together for cascading used washing liquid from one container to another container.

15. The industrial dishwasher of claim 1, wherein
the sanitizing station comprises a heater for maintaining fluid temperature of the sanitizing station to be about 50° C. to about 60° C.

16. The industrial dishwasher of claim 1, wherein
the neutralisation station comprises a prewashing container for mixing the cleaning agent with the sterilising agent.

17. The industrial dishwasher of claim 1, wherein
at least one of the detergent station, the sanitising station, and the neutralisation station comprises at least one stirrer for creating turbulence in at least one of the stations.

18. The industrial dishwasher of claim 1 further comprising
a rinsing station connected to at least one of the detergent station and the sanitising station for removing at least one of the cleaning agent and the sterilising agent.

19. The industrial dishwasher of claim 1, wherein
the neutralisation station, the detergent station, and the sanitising station are serially connected for washing the serviceware from the neutralisation station, to the detergent station and then to the sanitising station sequentially.

20. The industrial dishwasher of claim 1, wherein
at least one of the neutralisation station, the detergent station, and the sanitising station comprises a spray nozzle for discharging at least one washing fluid onto the serviceware.

21. The industrial dishwasher of claim 1 further comprising
a drying station for parching the serviceware.

22. The industrial dishwasher of claim 1 further comprising
a vision inspection station for examining washing quality of the serviceware automatically.

23. The industrial dishwasher of claim 1 further comprising
at least one steam station for discharging steam onto at least one of soiled serviceware and washed serviceware.

24. A method of using an industrial dishwasher, comprising:
providing a cleaning agent at a detergent station for washing serviceware;
providing a sterilising agent at a sanitising station for disinfecting the serviceware;
providing a neutralisation station; and
receiving and deactivating at least one of the cleaning agent and the sterilising agent by the neutralisation station.

25. The method of claim 24, further comprising
transporting soiled serviceware through the neutralisation station the detergent station and the sanitising station or a combination of any of these two or more stations sequentially.

26. The method of claim 24, further comprising
discharging electric current through an electrolyte for making at least one of alkali electrolysis water as the cleaning agent and acid electrolysis water as the sterilising agent.

27. The method of claim 24, further comprising
generating micro cavities in washing fluid at at least one of the detergent station, the sanitising station, and the neutralisation station.

28. The method of claim 24, further comprising
regulating fluid temperature of at least one of the detergent station, the sanitising station, and the neutralisation station to be lower than 65° C.

29. A method of making an industrial dishwasher, comprising:
providing a detergent station for washing serviceware with cleaning agent;
providing a sanitising station for disinfecting the serviceware with sterilising agent;
providing a neutralisation station connected to at least one of the detergent station and the sanitising station for deactivating at least one of the cleaning agent and the sterilising agent at the neutralisation station; and
providing at least one conveyor chain connected to at least one of the detergent station and the sanitising station for carrying the serviceware to the detergent station and the sanitising station.

30. The method of claim 29, further comprising
connecting an electrolyzer to the detergent station and the sanitising station.

31. The method of claim 29, further comprising
connecting a cavitation generator to at least one of the detergent station, the sanitising station, the neutralisation station, and the electrolyzer.
